(19) United States
(12) Patent Application Publication
(10) Pub. No.: US 2019/0216537 A1
(43) Pub. Date: Jul. 18, 2019

(54) PERCUTANEOUS INSTRUMENTS, AND MATERIALS, CONSTRUCTION AND METHODS OF USE

(71) Applicants: Adam E. M. Eltorai, Old Saybrook, CT (US); Damian E. Dupuy, Centerville, MA (US)

(72) Inventors: Adam E. M. Eltorai, Old Saybrook, CT (US); Damian E. Dupuy, Centerville, MA (US)

(21) Appl. No.: 15/871,707

(22) Filed: Jan. 15, 2018

(51) Int. Cl. A61B 18/14; A61B 18/18; A61B 17/17; A61B 17/34

(52) U.S. Cl. CPC A61B 18/1492; A61B 18/18; A61B 17/3403; A61B 17/3421; A61B 17/1703

(57) ABSTRACT

A percutaneous tool or device intended for image-guided placement or operation under medical imaging is fabricated with materials such as aluminum or aluminum alloys rather than stainless steel, or is otherwise configured to prevent beam hardening and the loss of low energy beam data during medical imaging that could otherwise degrade or produce confounding artifacts in the image. The improved tool, such as a percutaneous microwave ablation antenna or biopsy needle, can be more reliably and accurately positioned in relation to a targeted tissue site and thus operated more precisely and completely treat or sample a tumor or other tissue target in the body of a patient.

### BACKGROUND OF THE INVENTION

The invention related generally to medical imaging and to instruments and devices employed in image-guided percutaneous procedures.

Medical imaging underlies much current diagnostic and treatment activity. One imaging modality is computerized x-ray tomography (CT imaging), in which a region of the body is x rayed along multiple paths while an array of detector elements detects the received intensity, and the detected data is processed by mathematical transforms to computationally construct images of the tissue volume through which the x-ray scans have passed. CT images may present significantly greater detail than single-shot x-ray transmission images of soft tissue, and may be processed to more clearly show organs and differences in tissue regions. In radiographic imaging, contrast agents may also be administered to the patient to enhance the visibility of different soft tissues, and the incorporation of a disease-specific targeting agent into a contrast agent may also highlight specific diseased (e.g., cancerous) tissue or organ pathology present in the imaged region, thus providing a powerful diagnostic tool. For example, a nanoparticle-based image enhancer that is functionalized to bind to and accumulate on hepatocellular carcinoma cells, may be administered to a subject prior to imaging in order to selectively highlight images formed of the relevant region to reveal the presence of liver cancer. Other image-guided surgery may use ultrasound, endoscopy, magnetic resonance imaginary (MRI), functional magnetic resonance imaging (fMRI), positron emission tomography (PET) technologies, single photon emission computed tomography (SPECT_, X-ray fluoroscopy. C-arm CT, and optical imaging

A great variety of such specialized cancer-targeting or specific tissue-targeting agents have been developed, and some of these offer the prospect of greatly improved, early detection of a tumor, or specific identification of very small tumors or metastases. Other image enhancement agents may be used to delineate surrounding or contiguous tissues, such as vasculature, that are potentially relevant to planning a surgical or treatment intervention, or to refining an initial diagnosis, for example to distinguish a non-malignant cyst from a malignant tumor, or to discern boundaries between non-malignant tissue anomalies and malignancies requiring treatment.

Imaging studies are also important in preparing and staging surgical interventions, and when a tumor is present, early detection allows treatment at an early stage and reduces the likelihood of metastasis or recurrence. However, even when an appropriate targeting enhancement agent is available, its uptake by the targeted tissue or tissue state may be irregular, leading to incomplete visualization of the region of interest, or inability to determine the precise boundaries of a tumor. Moreover, features of some tissue anomalies, such as cysts, introduce artifacts into the resultant images that may confound proper visualization of tissue structures or their margins.

When imaging studies reveal presence of a malignancy, a local intervention which avoids subjecting the patient to whole-body or systemic chemotherapy, can be the treatment of choice. One local treatment is focused radiation, applied externally, wherein controlled relative movement of the patient's body and the treatment beam produce a stationary region centered on the treatment site that delivers higher, cell-killing, levels of energy to the tumor. Other local treatments may involve a percutaneous intervention, such as placement of a radioactive needle or slow-release chemotherapy drug in the tumor itself. More recently localized percutaneous thermal ablation, such as RF or microwave ablation, has offered treatment advances. Percutaneous microwave ablation involves positioning a microwave ablation needle/antenna in or near the tumor and actuating the antenna to heat and ablate contiguous tissue. Percutaneous microwave ablation, as well as percutaneous biopsy and other associated procedures, each require the penetration and/or insertion of a handpiece-manipulated device with a shaft-like or elongated support structure or applicator, and frequently employ image-guided positioning to accurately position the tip or a central stylet for sampling, or for ablating, or otherwise treating the target site. Such procedures include, by way of example and not limitation, fine-needle aspiration, core biopsy, needle localization, agiography, cholangiography, balloon angioplasty, endovascular aneurysm repair, embolization, thrombolysis, IVC filters, dialysis, TIPS, endovenous laser treatment, biliary intervention, central venous catheterization, drainage catheter placement, radiologically inserted gastronomy, chemoembolization, radioembolization, radiofrequency ablation, cryoblation, microwave ablation, percutaneous nephrostomy ureteral stent exchange, and vertebroplasty. Other procedures performed under flouroscopic guidance may include, facet blocks, medial branch blocks, rhizotomy, nerve root blocks, sacroiliac joint blocks, epidural injections, discograms, myelograms, and arthrograms. Other procedures performed under ultrasound guidance may include shoulder paralabral cyst aspiration, knee popliteal cyst aspiration, foreign body localization, and platelet rich plasma injection. Other procedures performed under computed tomography (CT) guidance, may include piriformis injection, iliopsoas bursa aspiration and collection aspiration and drainage.

Image-guided tumor ablation (IGTA) (e.g., microwave, radiofrequency, and cryosurgery) is a percutaneous procedure used to treat nonsurgical, solid-organ tumors. IGTA is an effective and repeatable therapy that can reduce and eradicate malignancies while preserving surrounding healthy tissue. The ablation instruments can be similar in size, shape and materials of construction to other kinds of percutaneous instruments. Most ablation applicators are made of stainless steel shafts of varying thicknesses and diameters. When placing the metal applicators under CT scan guidance, beam hardening artifacts can occur at the interface between substances of markedly different attenuations. The artifacts can make it difficult to see the tumor margin and its relationship to the tip of the ablation applicator. If the interventional radiologist or oncologic surgeon is unable to ablate the tumor completely to its edges, cancer remains in the body. Thus, visualizing of the borders of a tumor is essential for improved patient outcomes. Some recent studies have begun to recognize the clinically significant effects of image artifact on tumor ablation as well as the need for improved visualization. See, for example, Stattaus J, Kuehl H, Ladd S, Schroeder T, Antoch G, Baba H A, Barkhausen J, Forsting M. CT-guided biopsy of small liver lesions: visibility, artifacts, and corresponding diagnostic accuracy. Cardiovasc Intervent Radiol 2007; 5:928-935. Wang Z, Aarya I, Gueorguieva M, Liu D, Luo H, Manfredi L, Wang L, McLean D, Coleman S, Brown S, Cuschieri A. Image-based 3D modeling and validation of radiofrequency interstitial tumor ablation using a tissue-mimicking breast phantom. Int J Comput Assist Radiol Surg. 2012 November; 7(6):941-8. McWilliams S R, Murphy K P, Golestaneh S, O'Regan K N, Arellano R S, Maher M M, O'Connor O J. Reduction of guide needle streak artifact in CT guided biopsy. J Vase Intery Radiol. 2014 December; 25(12):1929-35.

Percutaneous microwave ablation is a highly efficient and highly localized hyperthermic treatment process that is capable of quickly raising local tissue temperatures to or above 60° C. for ablating a tumor. The shape of the actual treatment region depends upon the design and dimensions of the antenna, and may be an almond-, olive-spherical or oblong-shaped region about the end of the microwave antenna, corresponding to the microwave radiation pattern and the microwave absorption depth in tissue. Different antenna constructions provide somewhat different profiles of this effective ablation region, depending on the shape and dimensions of the antenna as well as its design—monopolar, bipolar or triaxial constructions—which may affect the launching, back-reflection or other aspects of the shape and energy efficiency of microwaves in tissue. (See FIG. 5). Furthermore, the probe may have a support or delivery tube in which a coolant circulates to counteract heating of the leads or conductors and avoid unwanted tissue damage outside the target region. Placement of such percutaneous devices has been performed using ultrasound (US) imaging for image guidance to aid positioning relative to a target vessel, tissue or tumor in accordance with accepted protocols. However, for safely and effectively targeting larger or ultrasound “occult” tumorous regions within or proximate to a vital organ or other sensitive tissue, x-ray CT imaging may be advantageous.

However, the presence of a metallic article or electrically conductive metal portion(s) of a treatment instrument such as wires, antenna needle, support member or delivery sheath, in a region undergoing CT x-ray imaging may cause beam hardening, and can be expected to introduce various artifacts into the x-ray image that confound diagnostic utility or the accuracy of positioning in relation to the target tissue and/or that otherwise alter the appearance of the target tissue in a confounding manner. Beam hardening may be especially likely with low-energy low dose x-rays commonly used for operating room imaging, and the effects may be increased when multiple closely-spaced probes are employed to define a larger, more complete, tissue ablation zone.

Beam hardening is the relative depletion of lower-energy x-rays from the beam, and it can occur during scans that encounter thick tissue (for example, through-body imaging of a deep target), or encounter hard tissue such as bone, or certain metal inclusions. In CT images, beam hardening and scatter both produce dark streaks between two high attenuation objects (e.g., metal or bone), with surrounding bright streaks. These image artifacts can be reduced using iterative reconstruction. Beam hardening caused by the thickness or orientation of tissue also causes pseudo-enhancement of certain tissue features such as the edges of renal cysts. In a variety of contexts, beam hardening artifacts in CT images can confound image interpretation.

Certain recognized beam hardening image artifacts may be corrected or reduced by various techniques such as positioning the x-ray source to avoid passing by bones or metal implants; modulating the beam power differently for different regions of the scan to correct for variation in tissue thickness; pre-hardening the beam to largely eliminate the contribution of low energy photons and thereby reduce the anomalies that would otherwise occur from the absorption of these photons in tissue; using dual energy CT; or certain iterative processing procedures such as the metal deletion technique (MDT). However, CT scanners are complex systems, and when an irregular metal percutaneous instrument or probe is to be deliberately introduced into the x-ray image field for image-guided positioning close to a tissue target, the existing techniques for reduction of hardening artifacts may be lengthy, difficult or cumbersome to set up or implement for the x-rays used image the probe as it is being positioned, and may result in sub-optimal imaging of the target tissue.

Similar treatments and technologies are used, with lesser extent, but growing, in veterinary medicine as well.

It would therefore be desirable to correct or avoid the introduction of beam hardening artifacts in medical imaging that are taken for performing percutaneous procedures and/or positioning an instrument during such procedures and to reduce instrument-induced artifacts during image-guided procedures.

### SUMMARY OF THE INVENTION

The problem of instrument-induced beam hardening and image artifacts thereof at normal diagnostic x-ray CT beam energies is addressed in accordance with the present invention by providing a percutaneous interventional tool, such as a microwave or other ablation probe or a percutaneously operated embolization, biopsy or other instrument, wherein a relevant portion of the instrument is fabricated with a low atomic number material to reduce or minimize the effect on lower energy x-rays by the instrument and thereby reduce beam hardening and avoid the introduction of beam hardening image artifacts. The relevant portion of the instrument may for example, be a tube, cannula or sheath used for introduction of, or for the support of an ablation antenna, or used as an outer antenna element; may be an electrical conductor or wire portion; and/or may be another portion of the instrument or antenna that lies in the imaging-induced field, such as an x-ray field. By selective use of low atomic number (low Z) materials, low dose CT images of enhanced fidelity are obtained at diagnostic levels of the x-ray beam energy.

### BRIEF DESCRIPTION OF THE DRAWING FIGURES

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 is a perspective view in visible wavelengths from a position outside the patient's body illustrating a plurality of cryoprobes inserted in a patient for treatment of a kidney cancer;

FIG. 2 shows a number of microwave ablation probes positioned about a target, as seen by the operator without CT visualization and before percutaneous insertion;

FIG. 2A shows microwave ablation probes positioned in a large liver tumor in a patient within a CT scanner;

FIGS. 3A-C are front views of three commercially available RF electrodes showing, respectively, an electrode with three straight needles, a first version of an electrode with deployable tines shown in the deployed state and a second version of an electrode with different deployable tines;

FIG. 4 is an illustration of another commercially available RF electrode;

FIG. 5A is an illustration of an exemplary monopole antenna of an electrode;

FIG. 5B is an illustration of an exemplary dipole antenna of an electrode;

FIG. 5C is an illustration of an exemplary slot antenna of an electrode;

FIG. 5D is an illustration of an exemplary sleeve antenna of an electrode;

FIG. 5E is an illustration of an exemplary triaxial antenna of an electrode;

FIG. 5F is a side perspective view of an exemplary active region of an antenna of an electrode;

FIG. 5G is an illustration of minimally invasive choke antenna of an electrode;

FIG. 6A is a CT image of prior-art probes illustrating artifacts produced in the CT image by interference with the beam;

FIG. 6B is a CT image of probes illustrating reduced artifacts produced in the CT image by interference with the beam with a probe of an embodiment of the present invention;

FIG. 6C is yet another CT image of prior-art probes illustrating artifacts produced in the CT image by interference with the beam;

FIG. 7 is a perspective view of a biopsy needle;

FIG. 8 is an illustration of an exemplary embodiment of an instrument having a segmented portion having a lower atomic number material;

FIG. 9 is an illustration of an exemplary embodiment of an instrument with a body made of a lower atomic number material;

FIGS. 10A and 10B are illustrations of an exemplary embodiment of an instrument with an antenna made from a standard material with a coating of a material with a lower atomic number over a portion or whole of the instrument;

FIGS. 11A and 11B are illustrations of an exemplary embodiment of an instrument with an antenna made from a standard material with a sheath of a lower atomic number material slide over a portion or whole of the instrument; and

FIG. 12 is an illustration of an exemplary embodiment of an instrument made from a standard material with a material of a lower atomic number embedded or integrally formed in a distal portion of the instrument.

### DESCRIPTION OF THE INVENTION

The invention and its advantages will be discussed with reference to a percutaneous microwave ablation probe or antenna.

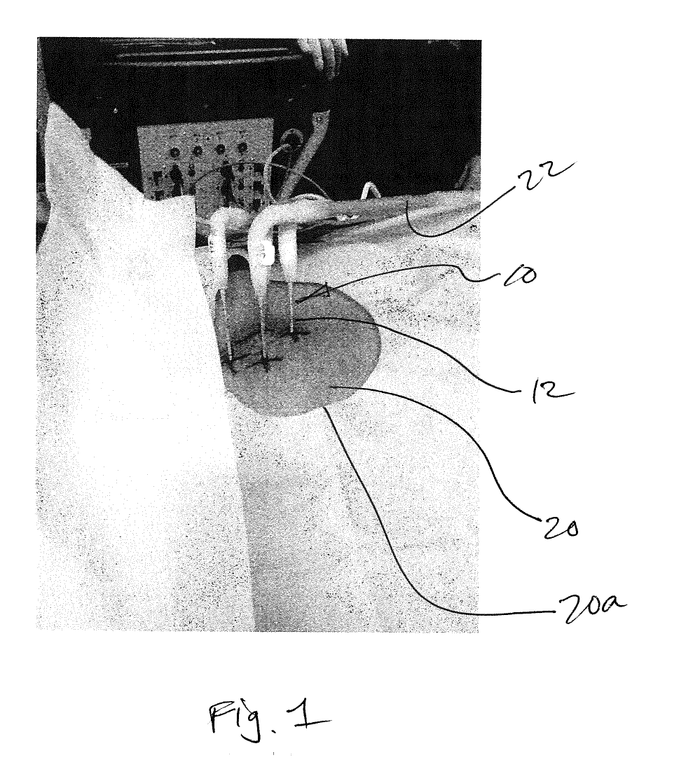

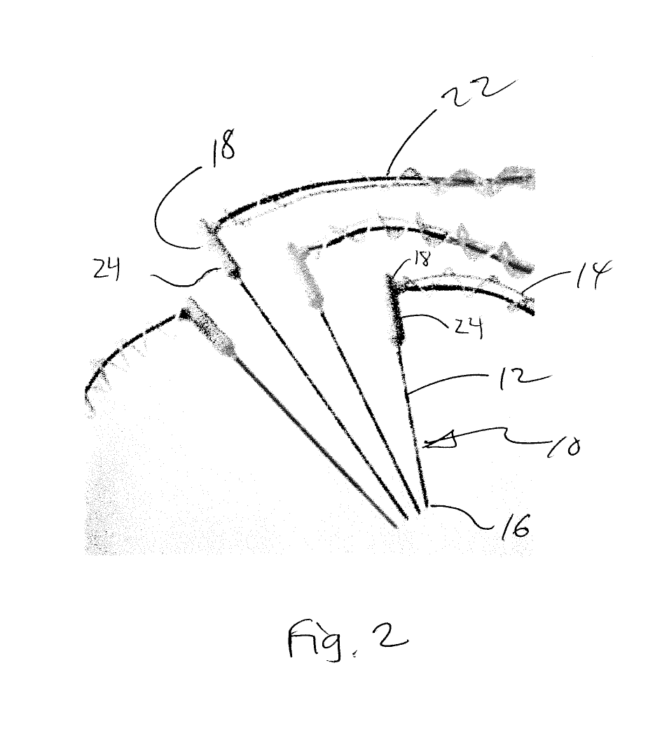

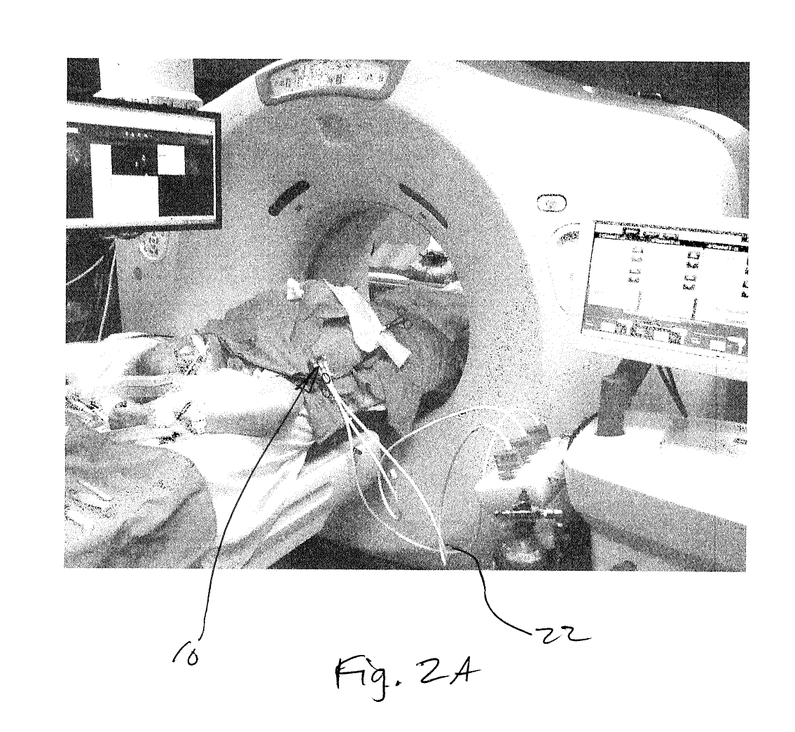

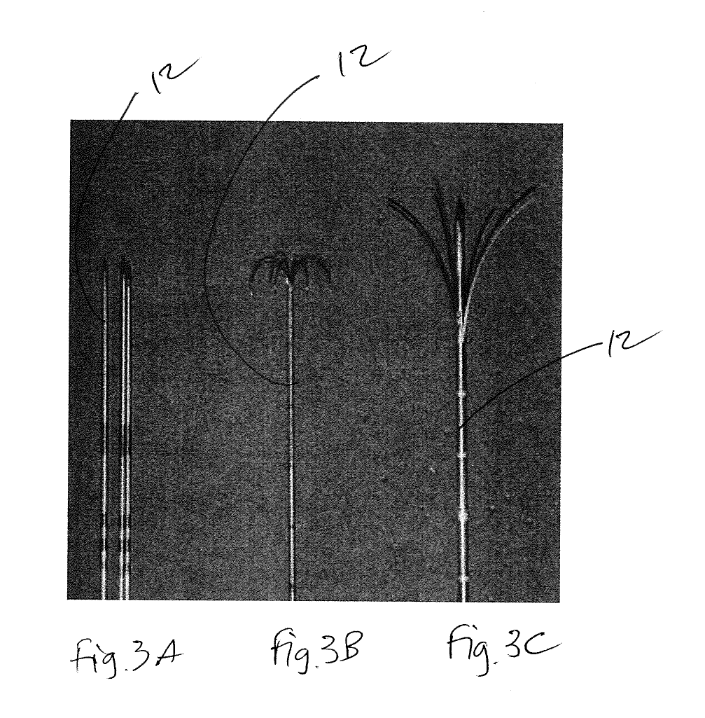

In accordance with the present invention, as seen in FIGS. 1 and 2, a microwave ablation antenna 10 may include a single needle-like conductor 12, a pair of spaced wires 14, or other appropriately shaped microwave-radiating antenna extending at the end of lead-in or support structure 18 for forming a characteristic tissue-heating microwave field at the tip 16 of the instrument. The antenna 10 may be a few centimeters in length to locally radiate microwave energy into the tissue 20 and quickly and controllably elevate tissue temperature and thus destroy tissue 20 in a region, such as an oval or almond-shaped region of tissue 20 located about the ablation antenna 10. Typically, the wire-like antenna 10 extends at the end of a flexible power-conducting coaxial cable or a jacket or metal tube 22 that connects to and is moved by and controlled by a hand piece 24 connected to the other end and formed with the support structure. In various devices, the antenna 10 with its cable assembly may itself be advanced within an introducer sheath like the ones used for biopsy needles, and the sheath may itself form a component of a shield, or a cooling system, or an electrical portion of a microwave antenna structure.

Cryoablation probes may have similarly shaped components, including, for example, a needle-like overall profile, and metallic portions for the cryogenic gas or fluid delivery, cooling nozzle at the tip, and/or return jacket. Similarly, RF ablation probes may be formed with a larger-diameter external jacket or trocar of stainless steel or the like, with an (electrically) uninsulated portion that serves as a ground for an array of RF treatment electrodes that deploy centrally therefrom.

In use, a percutaneous microwave ablation tool 10 may be positioned by image-guided insertion to a location near a tumor or intended tissue 20 treatment site, and then energized for a defined time period to heat and destroy tissue in a small region surrounding the antenna tip 16 in which microwave energy is substantially absorbed and causes ablative heating. The size and shape of the ablation region is largely determined by the specific antenna structure, the characteristic tissue microwave absorption coefficient, and the power and duration of actuation. The size and nature of an intended treatment site 20a may require insertion of several microwave antennas 10 with their axes offset a centimeter or more to define an effective treatment region larger than the range of a single antenna 10 so as to achieve effective thermal ablation of the entire target.

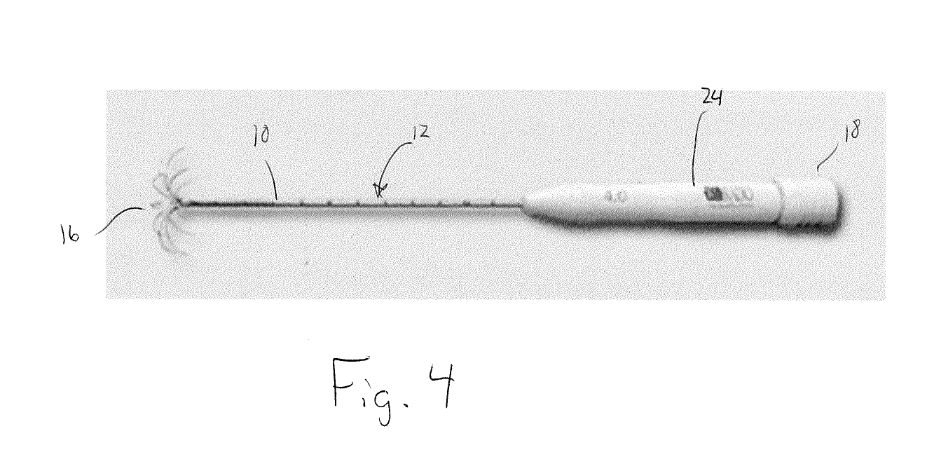

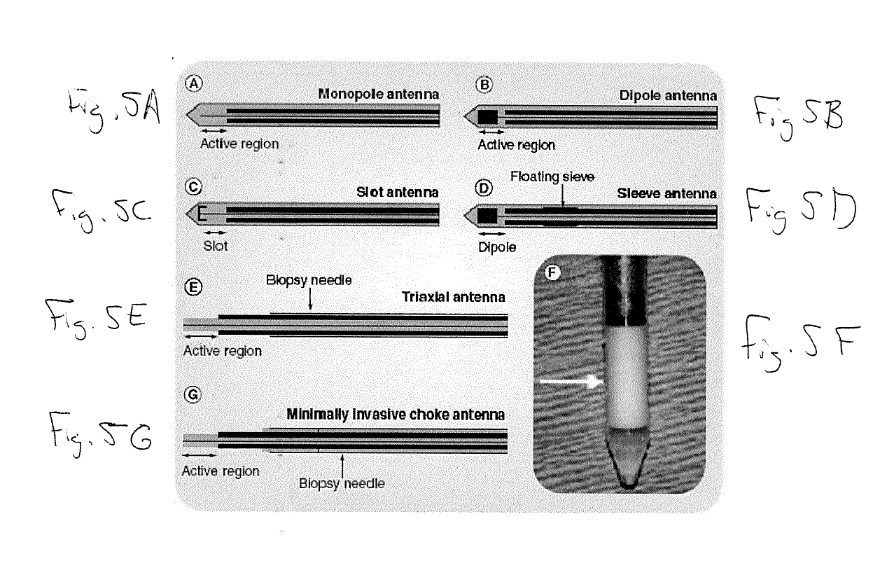

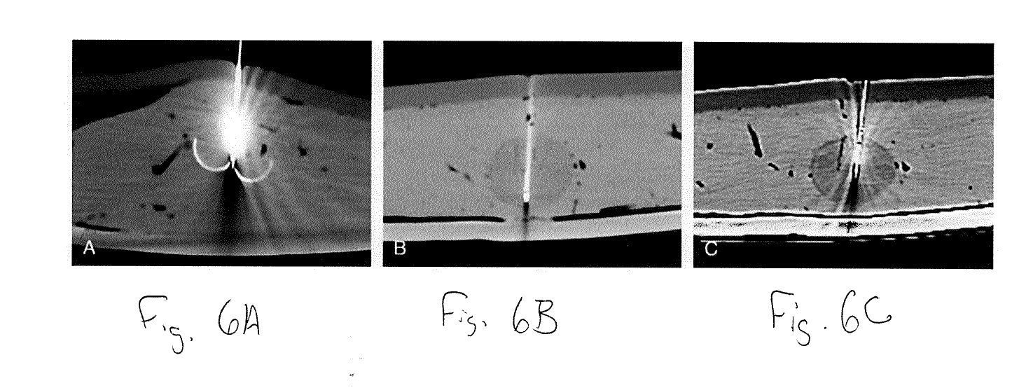

Placement and control of the needle or antenna 10 using a hand-held probe (see, e.g., FIG. 4) may involve first identifying the desired target region in study images, such as CT images, and may possibly also require fastening or placing a number of clips or markers (not shown) in or about the intended treatment site 20a to serve as registration points for accurate navigation of the treatment device in relation to the target region 20a. Then, as the surgeon, radiologist or technician moves the needle or antenna 10, its movement and placement are observed, for example using low-dose x-ray CT imaging. It may also be desirable to re-observe the tissue 20 that is being treated at one or more intervals during or after the ablation or as treatment progresses, to gauge the boundaries of the tumor or of the effectively treated region.

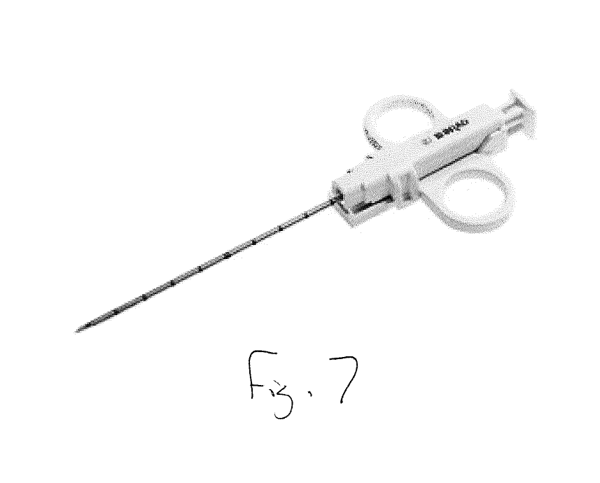

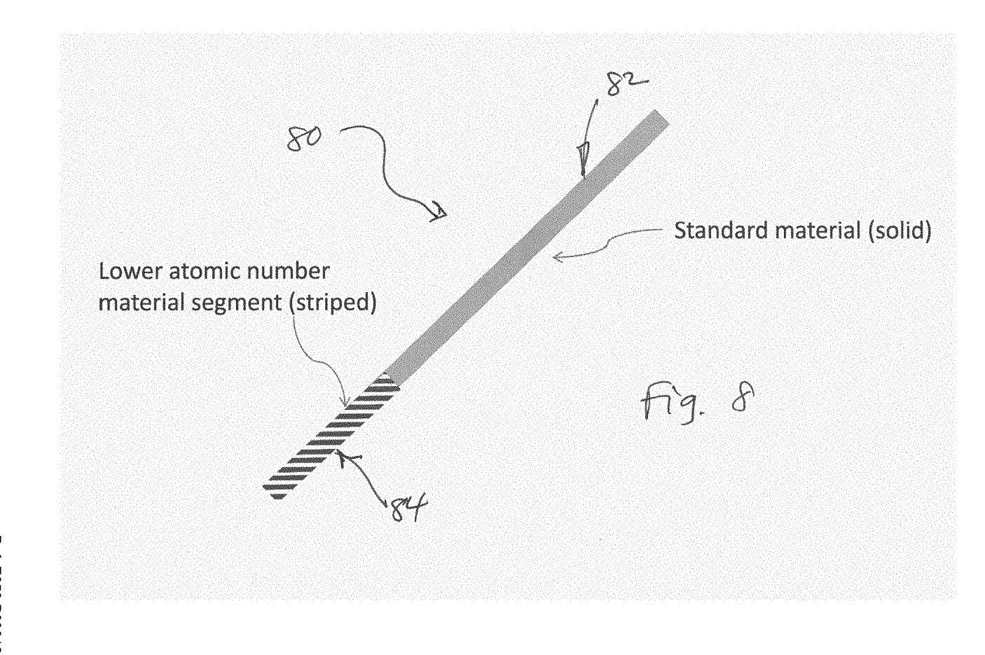

However, the presence of a metallic article or electrically conductive metal portions of a treatment instrument such as wires, antenna needle 12, support member or delivery sheath, in a region undergoing CT x-ray imaging may cause beam hardening, and can be expected to introduce various artifacts into the x-ray image that confound diagnostic utility or the accuracy of positioning in relation to the target tissue and/or that otherwise alter the appearance of the target tissue in a confounding manner. (See FIG. 6A-6C). Beam hardening may be especially likely with low-energy low dose x-rays commonly used for operating room imaging, and the effects may be increased when multiple closely-spaced probes are employed in the tissue. This is of particular concern in the arrays of cryoablation and microwave ablation probes 10 shown in FIGS. 1 and 2A, which are readily found in such a CT setting in the operating room.

The metal portions of instruments such as biopsy instruments (see, e.g., FIG. 7) or ablation devices 10 have often conventionally been formed of nickel, or of a stainless steel, which is typically an alloy of iron, cobalt, nickel and other high atomic number (high Z) elements. For example, needle-like portion 12 of the antenna 10 is one such component that is typically made of these materials. Stainless steels, and these alloy components, strongly absorb lower energy x-rays and result in beam hardening.

However, in accordance with the present invention, the metals used for constituting structures of the percutaneous instrument 10 are selected to be primarily low Z metals which do not harden the x-ray beams used for imaging or positioning the instrument or its operation. These preferred metals and electrically conductive components may be metals that are not conventionally used in surgical instruments or implants, for example they may include aluminum, as well as alloys thereof, such as aluminum-beryllium alloys and other such alloys, and these are selected or possibly developed as a new alloy, for their very low absorbance of x-rays. Some suitable alloys for these instrument components may be readily identified by persons skilled in the art, based on the prior certification of such an alloy for other medical uses, such as in bone pins, plates, dental or orthopedic implants such as joints, or based upon existing uses of the alloy in other implanted devices such as pump or pacemaker housings. Others may be newly developed based on the low-Z selection criterion and then experimentally varied to determine optimal alloy percentages and methods of preparation.

Even when a specific alloy may have been previously found to be unsuitable for some surgical applications, such as fixation pins or plates, due to adverse aging effects or the possibility of leaching or toxicity in the body, an alloy or low-Z metal may still be suitable for construction of the improved percutaneous instruments of this invention if it possesses necessary strength, conductivity, flexibility or other properties appropriate for substitution in and operation in the percutaneous instrument. This is because a percutaneous instrument that would reside in the body for only a short time, e.g. under one hour, would not be expected to degrade or cause extensive unwarranted emissions. This is particularly true for the needle-like portion 12 of antenna 10. Furthermore, other components used to alloy aluminum or beryllium need not be limited exclusively to low-Z atoms, because when an alloy only has a small concentration of a higher atomic number metal such as nickel or copper, the effect of this larger atom on x-ray hardening or energy-dependent absorption may also be relatively small.

Thus, a great number of new alloys may be found suitable for this non-hardening percutaneous instrument construction. Moreover, because leaching, corrosion or toxicity are not likely to be a concern for brief percutaneous usage, certain more reactive but conventionally ignored metals may be found suitable for fabricating non-beam hardening instruments. Corrosion effects may be reduced further if necessary by coating the metal surface to prevent interactions with body tissue or fluids. Moreover, this relaxation of parameters applies with greater force to single-use of dispensable components and accessories.

Thus, low-Z metals and alloys substantially consisting of a low-Z metal are deemed suitable candidates for fabrication of percutaneous instruments, including microwave ablation instruments and portions thereof, in accordance with the present invention and shown in the figures.

In addition to such metals, the invention contemplates forming a microwave ablation or biopsy instruments for percutaneous procedures, and/or associated system components, with non-metallic components, e.g., made of materials such as graphite, fiberglass or various polymers and combinations thereof. In that case the components are selected to have a required level of structural stiffness or strength and/or electrical conductivity appropriate to their function, but to produce little or no beam hardening.

With instrument-produced beam hardening actually reduced or even eliminated navigation and placement of the endoprobe under x-ray CT is improved even at typical diagnostic beam settings. It is further contemplated that by reducing beam hardening artifacts in this manner, one is then able to compare, and thus detect and quantify the relative effects of metal thickness, shell dimension or needle gauge, and other purely structural parameters of conventional probes on hardening and resultant images. Moreover, when instrument-induced hardening is removed, other imaging artifacts may be accurately measured explored or corrected using existing artifact correction protocols without the complications introduced by irregularly distributed instrument bodies in the x-ray imaging field.

In addition to redesign, or low-Z replacement of the components responsible for instrument-based hardening, the invention further contemplates substituting instruments of reduced thickness or hardening cross-section to reduce hardening effects in the x-ray paths and thereby decrease the amount of hardening or artifact that occurs. Thus, where the introducer is a 22 gauge biopsy introducer, or a similar gauge power cable: or where the antenna is a 14-1 gauge antenna, artifact reduction can be expected simply by forming these components of the same metal, but of a smaller dimension or gauge size. Metal-containing coupling structures or microwave tuning or launching structures located near the tip of the instrument can also be reconfigured to avoid or reduce their contribution to beam hardening. Hardening reduction can also be achieved by certain modifications of operating procedure, such as retraction or withdrawal of a metal introducer sheath before final imaging, or before performing post-ablation imaging, in order to acquire more accurate record documentation of the treatment site or scope.

In addition, while the discussion herein might be understood to advocate replacing metal tube, sheath or support structure with low-z metal or with non-metal structures, the use of metal such as aluminum is further contemplated for forming electrical conductors, similar to the conductive paths in flexible circuit boards, in which thin, flat metallized ribbons of foil on or between layers of polymer are used to interconnect power and load devices.

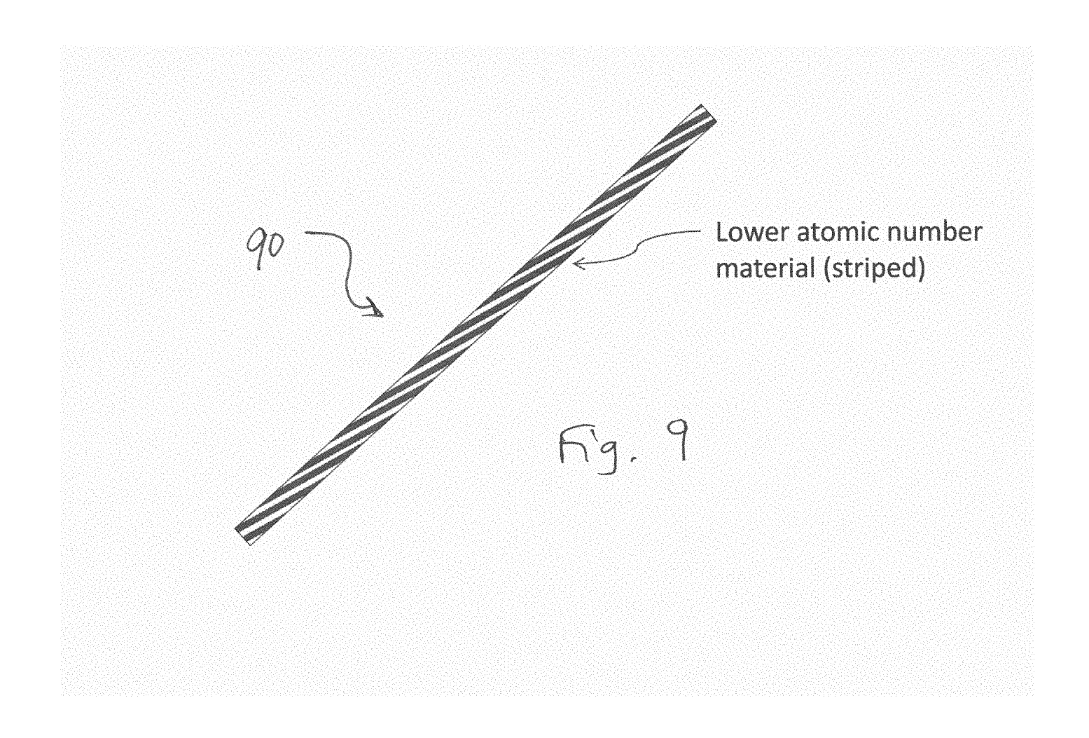

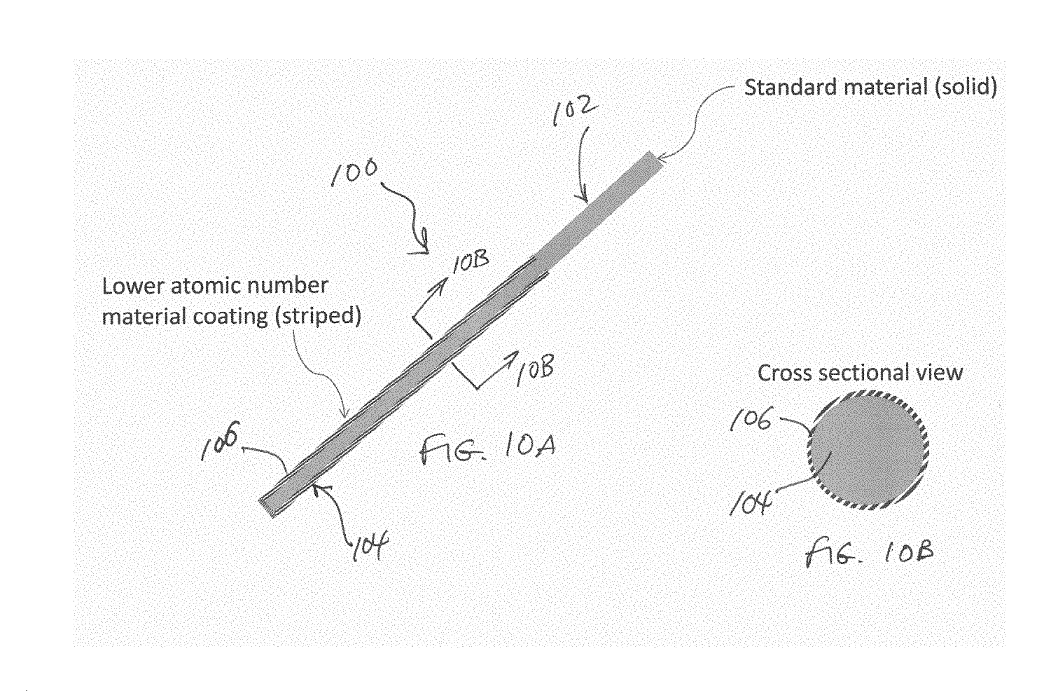

For instance, FIG. 8 shows an exemplary instrument 80 having a proximal end 82 and a needle-like distal end 84, where the proximal end 82 may be made of standard materials and the distal end 84 made from low-z metal or with non-metal structures. The distal end 84 is inserted in the patient where imaging resolution is critical. In another exemplary embodiment at FIG. 9, the entire instrument 90 is made from low-z metal or with non-metal structures.

Referring to FIGS. 10A and 10B, in another exemplary embodiment 100, an instrument having a proximal end 102 and distal end 104 is shown where the distal end 104 is coated with a low-z metal or with a non-metal material 106. The coating reduces image interference by the underlying standard material of the distal end 104 of the instrument.

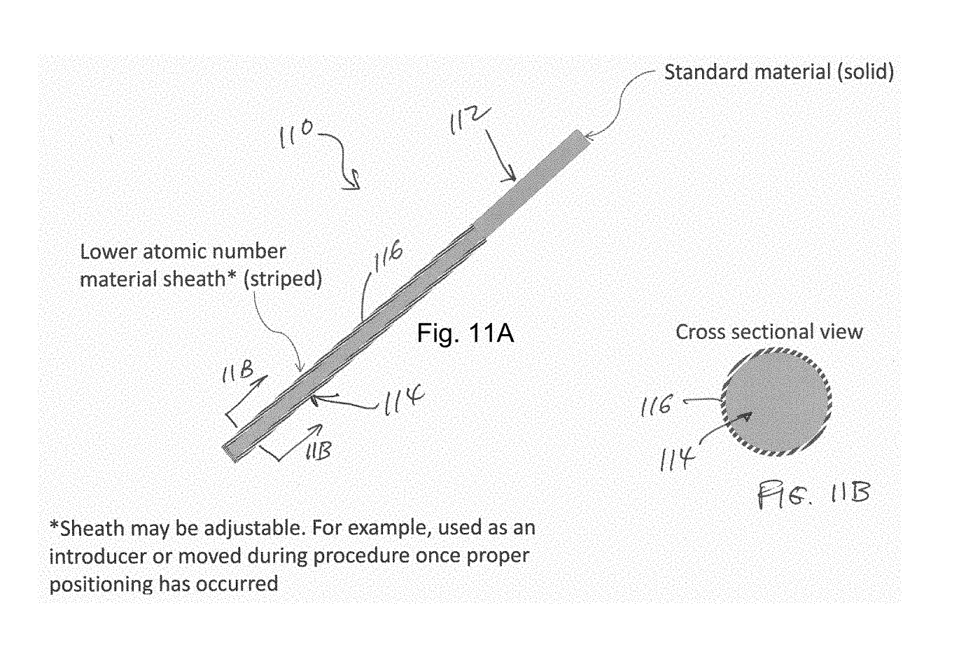

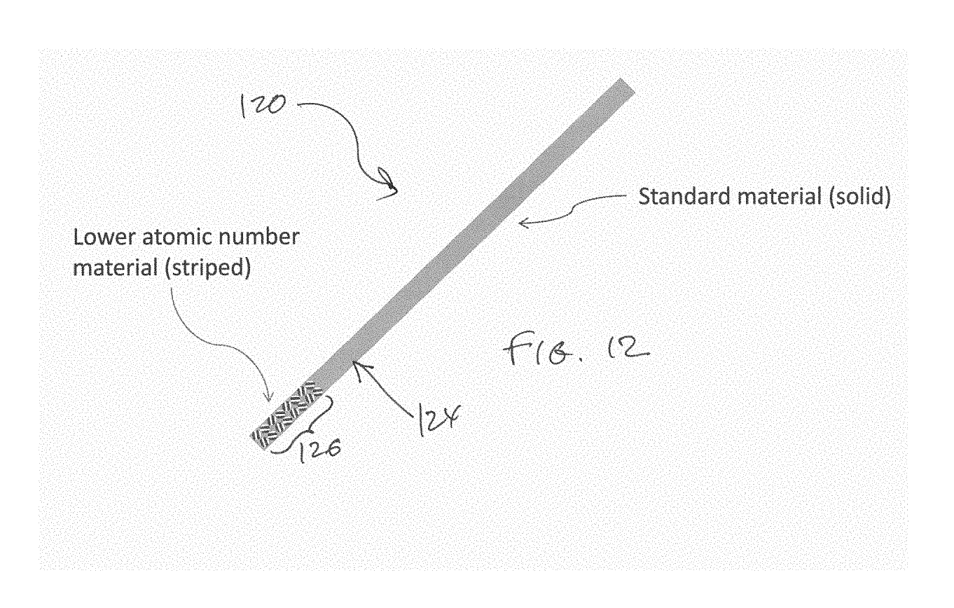

Referring to FIGS. 11A and 11B, in another exemplary embodiment 110, an instrument having a proximal end 112 and distal end 114 is shown where the distal end 114 is covered with a sheath 116 comprising a low-z metal or with a non-metal material. The sheath 116 reduces image interference by the underlying standard material in the distal end 114 of the instrument.

Referring to FIG. 12, in another exemplary embodiment 120, an instrument having a proximal end 122 and distal end 124 is shown where the distal end 124 is integrally molded or formed with a low-z metal or with a non-metal material 126 dispersed therein. The dispersed material 126 in distal end 124 reduces the overall image interference by the standard material of the instrument.

Use of such substitutions based on these low-Z and low cross-section conductors can support and give rise to entirely new forms of shielded cable, coaxial microwave conductors, coaxial-to-antenna junction couplers and other fundamental circuit elements or operative portions of a percutaneous microwave ablation device.

Furthermore, it should be noted that if the design of a microwave ablation antenna 10 depends upon or requires a specific dimensionally sized structure for its operation, or if such size requirement has formerly ruled out a construction, the substitution of a low-Z metal component in accordance with the present invention will enable use of larger structures without impairing CT visibility. Such effects may usefully facilitate dimensioning the probe for radiation at a longer or shorter microwave wavelength, or for energizing an array of multiple active tip assemblies, or to create a triaxial or other high-power antenna for ablating highly perfused organs or tissue. Advantageously, by substituting low-Z materials, thicker wall structures or bigger wires may be employed without introducing image artifacts, and use of low-Z materials can also enable the addition of housing or other structural or accessory features, such as fluid tubing to cool the tissue or the electrodes during antenna operation without incurring image degradation. Addition or enlargement of a cooling structure may be particularly useful in multi electrode RF or in microwave units designed to carry higher power to extend the size of the ablation region and enable lengthier ablation intervals without incurring undesirable operating drawbacks. In any of these circumstances, by eliminating metal-caused hardening artifacts one may expect to improve tumor margin visualization. The improved instruments may also permit one to set lower beam energies (60/80/100 KeV) with less severe instrument-dependent penalties and to employ other instrument settings if appropriate for the target tissue depth and location.

Experimentation with different scanning conditions can also determine optimal combinations of scan settings for a given applicator.

However, beyond applying conventional artifact correction processing, the inventors herein aim to reduce or eliminate the beam hardening introduced by the ablation device, replacing materials such as stainless steel by materials that are more transparent to x-rays.

The foregoing description depicts the context in which the invention operates and principles thereof as well as examples for a percutaneous microwave ablation instrument and representative embodiments. Further descriptions and form of the inventive improvements are delineated in the claims appended hereto, from which those skilled in the art will realize additional implementations and applications to other ablation tools, percutaneous delivery instruments, associated accessories and other devices, and all such variations and improvements are considered to be within the scope of the invention, as defined by the claims appended hereto and equivalents thereof.

What is claimed is:

1. An improved instrument for image-guided positioning or use in an imaged field of a medical imaging device, comprising:
an instrument having a component viewable by a device configured and arranged for of creating an image field; the component of the instrument being made of a material that reduces beam hardening thereby avoiding creation of undesired hardening artifacts in the imaged field.

2. The improved instrument of claim 1, wherein the component is a relevant conductive or structural metal portions of the instrument are made of low atomic number material or composition thereby prevent loss of low-energy beam data in the imaged field.

3. The improved instrument of claim 2, wherein the improved instrument is a percutaneous instrument with a material selected from the group consisting of: polymers, aluminum, aluminum alloys, low atomic number metals and other non-beam-hardening materials for the component.

4. The improved instrument of claim 1, wherein the instrument is a biopsy instrument, a tumor ablation instrument, or percutaneous image-guided instrument.

5. The improved instrument of claim 1, wherein the instrument is a percutaneous image-guided instrument with a metal sheath or introducer; the size or gauge of the metal sheath or introducer being reduced the size or gauge.

6. The improved instrument of claim 1, wherein the instrument is a microwave, RF or cryoablation tool, a biopsy tool, or any image-guided instrument

7. The improved instrument of claim 2, wherein the component is fabricated with aluminum or an alloy of aluminum to substantially reduce or avoid beam hardening.

8. The improved instrument of claim 2, wherein the component is fabricated with a low-Z metal to substantially reduce or avoid beam hardening.

9. The improved instrument of claim 2, wherein the instrument further includes electrical conductors or shielding; the conductors or shielding being made of a low-Z metal.

10. A method of treating a tumor, comprising the steps of:
percutaneously positioning an instrument near tissue using image-guided positioning;
actuating the instrument;
ablating or sampling the tissue at a site;
configuring and arranging the instrument to avoid beam hardening; and
whereby positioning with image guidance is precise and the site is more accurately imaged without introducing beam hardening imaging artifacts.

11. The method of claim 10, further comprising the step of:
further providing an introducer about the instrument;
retracting the introducer prior to image-guided positioning so as to form an image with enhanced quality having reduced beam hardening artifacts.

12. The method of claim 10, wherein the instrument is a treatment instrument or sampling instrument.

13. The method of claim 10, wherein the instrument is a microwave ablation antenna or biopsy needle.

14. A composition for manufacturing a medical device with low beam hardening properties, comprising:
a low atomic number material.

15. The composition of claim 14, wherein the low atomic number material is selected from the group consisting of polymers, aluminum, aluminum alloys, low atomic number metals and other non-beam-hardening materials for the component.

16. The composition of claim 15, wherein the low atomic material comprises aluminum-beryllium alloys.

17. A method of manufacturing a medical device with reduced beam hardening properties, comprising:
forming a portion of an instrument with a low atomic number material.

18. The method of claim 17, further comprising forming a portion of the instrument from a standard material.

19. The method of claim 17, wherein the step of forming a portion of an instrument with a low atomic number material comprises coating a portion of the instrument with a low atomic number material coating.

20. The method of claim 17, wherein the step of forming a portion of an instrument with a low atomic number material comprises forming a sheath with a low atomic number material coating sized and dimensioned to slide onto a portion of an instrument.

21. The method of claim 20, further comprising sliding the sheath of low atomic number material onto a portion of the instrument.

22. The method of claim 17, wherein the step of forming a portion of an instrument with a low atomic number material comprises integrally forming a portion of the instrument with a low atomic number material and a standard material.

23. A medical device with reduced beam hardening properties, comprising:
an instrument body having a proximal end and a distal end;
wherein at least a portion of the instrument body comprises a low atomic number material;
whereby the instrument, when used for image-guided positioning or used in an imaged field of a medical imaging device, produces fewer image artifacts.

24. The device of claim 23, wherein the distal end of the instrument body is formed from a low atomic number material.

25. The device of claim 24, wherein the proximal end of the instrument body if formed from a standard material.

26. The device of claim 23, wherein the entire instrument body is formed from a low atomic number material.

27. The device of claim 23, wherein at least the distal end of the instrument body is coated with a coating formed from a low atomic number material.

28. The device of claim 23, wherein at least the distal end of the instrument body is covered in a sheath formed from a low atomic number material.

29. The device of claim 23, wherein a low atomic number material is integrally formed in at least the distal end of the instrument body.

30. The device of claim 23, wherein the low atomic number material is selected from the group consisting of polymers, aluminum, aluminum alloys, low atomic number metals and other non-beam-hardening materials for the component.

31. The device of claim 30, wherein the low atomic material comprises aluminum-beryllium alloys.
